(19) United States
(12) Patent Application Publication
(10) Pub. No.: US 2004/0033488 A1
(43) Pub. Date: Feb. 19, 2004

(54) Single cell assessement of viral infection/replication

(76) Inventors: Omar Perez, Daly City, CA (US); Garry P. Nolan, San Francisco, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/370,649

(22) Filed: Feb. 19, 2003

Related U.S. Application Data
(60) Provisional application No. 60/358,425, filed on Feb. 19, 2002.
(60) Provisional application No. 60/359,153, filed on Feb. 20, 2002.

(51) Int. Cl. C12Q 1/70

(52) U.S. Cl. 435/5

(57) ABSTRACT

This invention relates to methods of separating virally infected viable cells from dead cells using antibodies specific for intracellular proteins and a covalent nucleic acid binding agent. The method can be readily adapted for assessing viral infection and/or replication in viable cells, identifying anti-viral agent, and monitoring anti-viral therapy

### CROSS-REFERENCE TO RELATED APPLICATIONS

This application claims priority to two pending U.S. provisional applications Ser. Nos. 60/358,425 and 60/359,153, filed on Feb. 19, 2002 and Feb. 20, 2002, respectively. These priority applications are hereby incorporated herein by reference in their entirety.

### STATEMENT OF RIGHTS TO INVENTIONS MADE UNDER FEDERALLY SPONSORED RESEARCH

This invention was made with Government support under contracts awarded by the National Institutes of Health, NIH2R01AI35304. The Government has certain rights in this invention.

### TECHNICAL FIELD

This invention is in the field of immunology and molecular pharmacology. Specifically, the invention relates to methods of separating virally infected viable cells from dead cells using antibodies specific for intracellular proteins and a covalent nucleic acid binding agent. The method can be readily adapted for assessing viral infection and/or replication in viable cells, identifying anti-viral agent, and monitoring anti-viral therapy.

### BACKGROUND OF THE INVENTION

Human immunodeficiency virus (HIV) has become a major worldwide epidemic. Since its discovery in 1981, HIV has killed over 19 million people with 3 million people dying in the year 2000 alone. According to the National Institute of Allergy and Infectious Diseases (NIAID), more than 6,500 young people aged 15 to 24 became infected with HIV each day in the year 2000. Further, the Joint United Nations Programs on HIV/AIDS reports that today over 36.1 million people are estimated to be living with HIV. In the U.S. alone, more than 765,000 cases of HIV infection have been reported to the U.S. Centers for Disease Control and Prevention (CDC) as of December 2001, with deaths totaling over 448,000 people. Acquired immunodeficiency syndrome (AIDS) caused by HIV infection is now the fifth leading cause of death in the United States among people aged 25 to 44, and approximately 40,000 new HIV infections occur each year in the United States.

By killing or damaging cells of the body's immune system, the virus causes acquired immunodeficiency syndrome (AIDS) and progressively destroys the body's ability to fight infections and disease. As the virus multiplies and kills immune cells, the body becomes vulnerable to opportunistic infections and other illnesses, ranging from pneumonia to cancer. The hallmark of HIV infection is the progressive decline in the blood levels of CD4+ T cells (also called “T-helper” cells), the immune system's key infection fighters. Given the threat of this widespread and deadly disease, there exists a significant need for therapies to combat HIV infection.

Despite ongoing improvement in our understanding of the disease, HIV infection has remained resistant to medical intervention. There is currently no cure for AIDS. Over the past 10 years, researchers have been investigating drugs to fight HIV infection. These include both nucleoside reverse transcriptase (RT) inhibitors that interrupt virus replication at an early stage, as well as protease inhibitors that interrupt virus replication at later stages in the viral life cycle. Researchers are investigating exactly how HIV damages the immune system to develop and test HIV vaccines and new therapies for the disease. This antiviral research requires evaluating the HIV virus and its effects on infected cells.

Traditional approaches to assessing viral infection include the use of bulk measurements, techniques to amplify detection, and flow cytometry. These techniques detect antibodies specific to viral antigens that are expressed in presence of the HIV. Such antigens include, but are not limited to, intracellular molecules such as p24, gp 120, rev tat, reverse transcriptase, HIV-1 protease, and HIV-1 integrase. While the use of bulk measurements and molecular techniques such as RT-PCR and p24 ELISA are useful in detecting intracellular antigens, they cannot quantitatively identify the viable cells harboring viral infection. Current methods employing flow cytometry or fluorescence activated cell sorting (FACS) are optimal for quantitatively assessing cell populations based on surface phenotype.

In flow cytometry, fluorescently labeled antibodies are used for single-cell detection of both surface (e.g., receptors) and intracellular antigens (e.g., p24, rev, tat, gp 120, reverse transcriptase, HIV-1 protease, and HIV-1 integrase). The technique is robust and amenable for high throughput screening of large populations of cells. Further, the flow cytometric platform allows for a multiparameter assessment of expression of viral antigens representative of a particular cell type, such as those infected with HIV-1. Detection of the labeled target molecules allows for isolation of infected cells and analysis which can then be used for diagnosis. FACS is also useful for cellular based assays such as cytotoxicity, apoptosis, and viability, among others. However, this technique has not been adapted to effect single cell assessment of viral infection or replication.

Presently, cell viability is routinely performed by membrane exclusion dyes such as propidium iodide (PI). If a cell's membrane has been compromised, it will stain with PI and is so labeled “dead.” PI, however, does not remain permanently attached during subsequent permeabilization steps. This results in false positive readings where non-viable or “dead” cells are identified as infected with an intracellular virus. Thus, while PI is useful for surface staining alone, it is inadequate for intracellular staining where the permeabilization conditions can cause reversible binding of PI and inadvertently label cells that are not dead.

An accurate assessment of the population of viable cells infected with the virus is of great significance in diagnosis and prognosis of AIDS. It is known that cells in HIV and other virus-infected patient undergo spontaneous apoptosis. Therefore, a significant percentage of cells may have died before the cells are processed for further analysis. In the process of screening anti-viral agents (e.g. anti-HIV agents) that induce death of viral infected cells, an assessment of the relative abundance of the infected viable cells and dead cells is crucial.

As mentioned above, there remains a considerable need for methods applicable for a single cell assessment of virally infected viable cells. In particular, there remains a need for a method of separating virally infected viable cells from dead cells using antibodies specific for intracellular proteins and a covalent nucleic acid binding agent. Such method should also be readily amenable for identifying anti-viral agent and monitoring anti-viral therapy. The present invention satisfies these needs and provides related advantages as well.

### SUMMARY OF THE INVENTION

A principal aspect of the present invention is the design of a technique that allows high throughput separation of virally infected viable cells from dead cells. The method employs antibodies specific for intracellular proteins that are expressed specifically in response to a viral infection, and a nucleic acid binding agent that recognizes preferentially dead cells. The method can be readily adapted for assessing viral infection and/or replication in viable cells, identifying anti-viral agent, and monitoring anti-viral therapy.

Accordingly, the present invention provides a method of separating virally infected viable cells from dead cells. The method comprises the steps of: (i) providing a population of cells from an individual suspected of having a viral infection; (ii) staining the population of cells with a covalent nucleic acid binding agent; (iii) contacting the population of cells of step (ii) with a labeled antibody specific for an intracellular protein that is expressed specifically upon viral infection of the cells, under conditions suitable for a specific binding of the antibody to the intracellular protein within a cell; and (iv) separating the population of cells of step (iii) to obtain a plurality of cells that are not stained with the covalent nucleic acid binding agent but are labeled with the antibody, and/or to obtain a separate population of cells that are stained with the nucleic acid binding agent.

In one aspect of this embodiment, the individual is suspected of having an HIV, EBZ or Ebola infection. In another aspect, the individual is suspected of having a Herpes virus infection. In yet another aspect, the individual is suspected of having Hepatitis virus infection which is mediate by one or more of the following viruses: Hepatitis A, Hepatitis B, Hepatitis C, and Hepatitis D.

This invention also provides a method of assessing HIV infection and/or replication in viable cells in an individual suspected of having an HIV viral infection. The method comprises the steps of: (i) providing a population of cells from the individual; (ii) staining the population of cells with a covalent nucleic acid binding agent; (iii) contacting the population of cells of step (ii) with a labeled antibody specific for an intracellular protein that is expressed specifically upon viral infection of the cells, under conditions suitable for a specific binding of the antibody to the intracellular protein within a cell; and (iv) separating the population of cells of step (iii) to obtain a plurality of cells that are not stained with the covalent nucleic acid binding agent but are labeled with the antibody, thereby assessing HIV infection of viable cells in the individual.

Also embodied in the present invention is a method of monitoring the effectiveness of an anti-viral therapy comprising assessing viral infection and/or replication in viable cells in an individual, wherein a reduction in viral infection and/or replication is indicative of the effectiveness of the therapy, wherein the assessment comprises the steps of: (i) providing a population of cells from an individual infected with the virus; (ii) staining the population of cells with a covalent nucleic acid binding agent; (iii) contacting the population of cells of step (ii) with a labeled antibody specific for an intracellular protein that is expressed specifically upon viral infection of the cells, under conditions suitable for a specific binding of the antibody to the intracellular protein within a cell; and (iv) separating the population of cells of step (iii) to obtain a plurality of cells that are not stained with the covalent nucleic acid binding agent but are labeled with the antibody, thereby assessing viral infection of viable cells in the individual.

Further provided in the present invention is a method of identifying an anti-viral agent, comprising assessing viral infection and/or replication in viable cells in an individual infected with the virus, wherein a reduction in viral infection and/or replication upon contacting a candidate anti-viral agent with a population of cells from the individual is indicative of the identification of an anti-viral agent, wherein the assessment of the viral infection and/or replication in viable cells in the individual comprises the steps of: (i) providing a population of cells from the individual; (ii) staining a population of cells obtained from the individual of step (i) with a covalent nucleic acid binding agent; (iii) contacting the population of cells of step (ii) with a labeled antibody specific for an intracellular protein that is expressed specifically upon viral infection of the cells, under conditions suitable for a specific binding of the antibody to the intracellular protein within a cell; and (iv) separating the population of cells of step (iii) to obtain a plurality of cells that are not stained with the covalent nucleic acid binding agent but are labeled with the antibody, thereby assessing viral infection of viable cells in the individual.

In practicing all embodiments of the present invention, preferred covalent nucleic acid binding agents include but are not limited ethidium monoazide (EMA) and actinomycin D. The antibodies specific for the intracellular protein can be labeled with fluorescent or radioactive moieties. The intracellular proteins can be proteins that are expressed specifically upon viral infection of the cells. Such proteins include those that are encoded by the virus and host cell proteins that are overexpressed in response to the viral infection. Preferred viral proteins include but are not limited to HIV proteins selected from the group consisting of p24, rev, tat, gpl2O, reverse transcriptase, HIV-1 protease, and HIV-1 integrase.

Any cells including animal and plant cells which are capable of being infected by viruses are contemplated. Preferred cells are mammalian cells, and preferably peripheral blood mononuclear cells. Whereas the cells stained with the covalent DNA are dead cells, the cells that are not stained with the nucleic acid binding agent but labeled with the antibody are virally infected viable cells. The separation of the virally infected viable cells and dead cells is effected by a cell sorting process, preferably by FACS.

### BRIEF DESCRIPTION OF THE DRAWINGS

FIG. 1 shows a flow diagram comparing one and two step fixation and permeabilization procedures. These procedures allow for intracellular staining and detection of cytoplasmic proteins.

FIG. 2 shows an example of single cell HIV-1 infection detection by intracellular p24 staining. IL-2 activated PBMC were infected with HIV (TCID₅₀=300/1×10⁶ cells), cultured for 6 days and stained for surface marker CD4, annexin-V, intracellular p24, and EMA. Live gated (EMA negative) cells were gated for p24 levels and correlated with CD4 and annexin-V markers.

FIG. 3 shows that HIV-1 infection and high concentrations of Gd-Tex lowers redox levels in vitro.

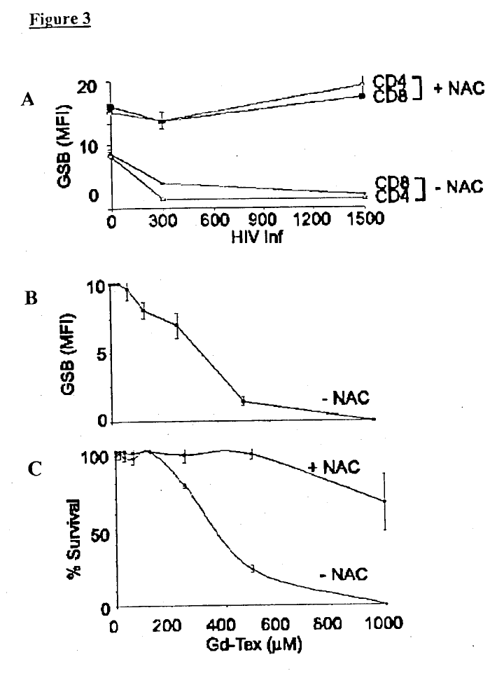

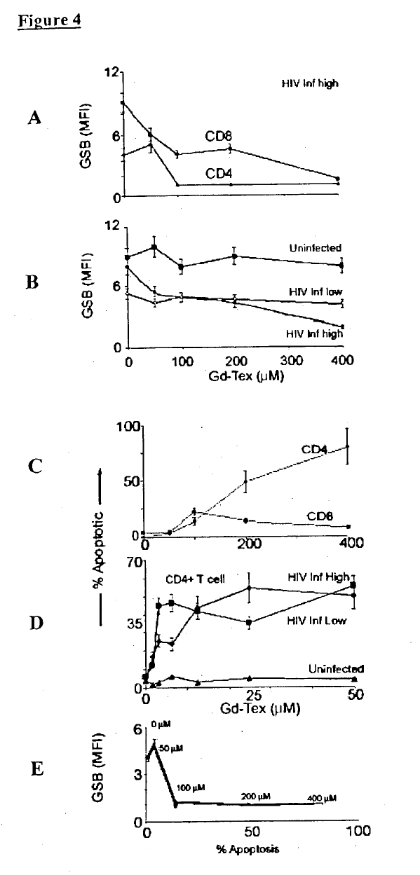

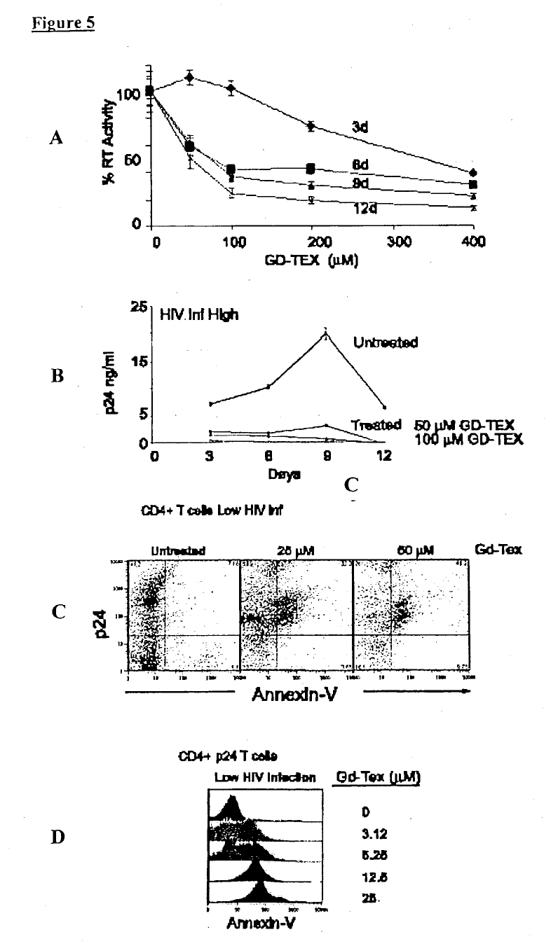

In FIG. 3A, the graph shows glutathione levels in uninfected, HIV-1 low multiplicity of infection (TCID₅₀=300/1×10⁶ cells) and HIV-1 high multiplicity infection (TCID₅₀=1500/1×10⁶ cells). PBMC were isolated and HIV-1 infected as described in materials and methods. Intracellular glutathione levels were assessed using monochlorobimane fluorescence and samples were analyzed by flow cytometry. Median fluorescence intensity (MFI) values for monochlorobimane fluorescence (gluthatione-s-bimane, GSB) of uninfected, and HIV-1 infected T cells (TCID₅₀ displayed on X-axis), and cells treated with and without NAC (5 mM, 24 hr).

FIG. 3B shows GSB levels of IL-2 activated PBMC as a function of Gd-Tex concentration.

FIG. 3C shows a Gd-Tex dose response curve on whole PBMC treated in the presence of absence of NAC (5 mM, 24 hr). Survival was determined using PI exclusion flow cytometry assay.

FIG. 4 shows that motexafin gadolinium cytotoxicity is enhanced in CD4 T cells infected with HIV-1.

FIG. 4A shows median glutathione levels of HIV-1 infected (TCID₅₀=1500/1×10⁶ cells) CD4 and CD8 T cells as a function of Gd-Tex concentration (post 6 days).

FIG. 4B shows median glutathione levels of uninfected, high HIV-1 infected and low HIV-1 infected CD4 T cells as a function of Gd-Tex treatment (post 3 days). Error bars denote standard deviation of 9 independent experiments from 12 healthy donors.

FIG. 4C shows that Gd-Tex induces apoptosis selectively in HIV-1 infected CD4 T cells. PBMC were infected with a high HIV-1 dose, incubated for three days with indicated concentrations of Gd-Tex, and assessed for annexin-V by flow cytometry. Live CD3 cells were gated and apoptotic percentages quantified for CD4 and CD8 cells.

FIG. 4D shows Gd-Tex induced apoptosis in live CD4 T cells infected with HIV-1. PBMC were infected with high and low HIV-1 doses and treated for six days with the indicated concentrations and processed as indicated above.

FIG. 4E shows that low median GSB is proportional to the induction of apoptosis as a function of Gd-Tex concentration. Median GSB values and percent apoptotic cells for CD4 T cells infected with HIV-1 (low) were plotted as a function of Gd-Tex concentration post 6 day treatment. Error bars denote standard deviations of at least three independent experiments.

FIG. 5 shows the inhibition of HIV-1 production by Gd-Tex.

FIG. 5A shows a reverse transcriptase activity assay of HIV-1 infected PBMC (TCID₅₀=300/1×10⁶ cells) as a function of Gd-Tex concentration and time. HIV-1 infected PBMC were incubated with Gd-Tex at the indicated concentration and cell-free supernatants were collected after 0, 3, 6, 9, and 12 days. Diluted supernatants were spotted in 96 well plates and reverse transcriptase activity was determined by RT activity assay kit (Molecular Probes) and previously using conventional radioactive RT activity measurements (data not shown). Values are normalized to HIV-1 infected Gd-Tex untreated cells.

FIG. 5B shows p24 levels over time as a function of Gd-Tex treatment for HIV-1 infection (TCID₅₀=300/1×10⁶ cells). p24 levels were determined by p24 ELISA and normalized to a p24 standard curve. Error bars denote standard deviation of at least three independent experiments from 9 healthy donors.

FIG. 5C shows an intracellular p24 stain of CD4 T cells infected with HIV-1 and treated with Gd-Tex at indicated concentrations for 6 days. Live CD4 T cells were gated and analyzed for annexin-V and p24 stain. Note decrease of p24 cells in the 50 μM treated culture, likely due to their depletion under Gd-Tex culture conditions.

FIG. 5D shows the titration of p24+ CD4 T cells with Gd-Tex . IL-2 activated, HIV-infected cultures were treated with Gd-Tex at the indicated concentrations for 6 days and processed for flow cytometry. Cells were gated for live p24 positive CD4 T cell and displayed for annexin-V stain. Histograms are representative of 4 independent experiments.

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure.

General Techniques:

The practice of the present invention will employ, unless otherwise indicated, conventional techniques of immunology, biochemistry, chemistry, molecular biology, microbiology, cell biology, genomics and recombinant DNA, which are within the skill of the art. See, e.g., Matthews, PLANT VIROLOGY, 3^{rd} edition (1991); Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Definitions:

As used in the specification and claims, the singular form “a,” “an,” and “the” include plural references unless the context clearly dictates otherwise. For example, the term “a cell” includes a plurality of cells, including mixtures thereof.

The terms “polypeptide”, “peptide” and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The term “antibody” as used herein refers to immunoglobulin molecules and immunologically active portions of immunoglobulin molecules, i.e., molecules that contain an antigen-binding site which specifically binds (“immunoreacts with”) an antigen. Structurally, the simplest naturally occurring antibody (e.g., IgG) comprises four polypeptide chains, two heavy (H) chains and two light (L) chains inter-connected by disulfide bonds. The immunoglobulins represent a large family of molecules that include several types of molecules, such as IgD, IgG, IgA, IgM and IgE. The term “immunoglobulin molecule” includes, for example, hybrid antibodies, or altered antibodies, and fragments thereof. It has been shown that the antigen binding function of an antibody can be performed by fragments of a naturally-occurring antibody. These fragments are collectively termed “antigen-binding units” ( “Abus”). Abus can be broadly divided into “single-chain” (“Sc”) and “non-single-chain” (“Nsc”) types based on their molecular structures.

Also encompassed within the terms “antibodies” and “Abus” are immunoglobulin molecules of a variety of species origins including invertebrates and vertebrates. The term “human” as applies to an antibody or an Abu refers to an immunoglobulin molecule expressed by a human gene or fragment thereof. The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

“Non-single-chain antigen-binding unit” (“Nsc Abus”) are heteromultimers comprising a light-chain polypeptide and a heavy-chain polypeptide.

Single-chain antigen-binding unit” (“Sc Abu”) refers to a monomeric Abu.

Although the two domains of the Fv fragment are coded for by separate genes, a synthetic linker can be made that enables them to be made as a single protein chain (i.e. single chain Fv (“scFv”) as described in Bird et al. (1988) Science 242:423-426 and Huston et al. (1988) PNAS 85:5879-5883) by recombinant methods.

A “covalent nucleic acid-binding agent” refers to natural and synthetic compounds that are capable of covalently binding to nucleic acids.

The term “monoclonal antibody” as used herein refers to an antibody composition having a substantially homogeneous antibody population. It is not intended to be limited as regards to the source of the antibody or the manner in which it is made. Monoclonal antibodies are highly specific, being directed against a single antigenic site. In contrast to conventional (polyclonal) antibody preparations which typically include different antibodies directed against different determinants (epitopes), each monoclonal antibody is directed against a single determinant on the antigen.

“A population of monoclonal antibodies” refers to a plurality of heterogeneous monoclonal antibodies, i.e., individual monoclonal antibodies comprising the population may recognize antigenic determinants distinct from each other.

An antibody “specifically binds to” or “specific for” an antigen (e.g. an intracellular protein) if the antibody binds with greater affinity or avidity than it binds to other reference antigen including polypeptides or other substances.

“Antigen” as used herein means a substance that is recognized and bound specifically by an antibody. Antigens can include peptides, proteins, glycoproteins, polysaccharides and lipids; portions thereof and combinations thereof.

“Mammals” are vertebrate animals which are characterized by giving live birth to their young and having hair on their bodies. As used herein, “mammals” include, but are not limited to, rabbits, murines, simians, humans, farm animals, sport animals, and pets.

A “subject,” or “individual” is used interchangeably herein, which refers to a vertebrate, preferably a mammal, more preferably a human.

As used herein, the term “HIV-specific immune response” is intended to include a positive or negative immune response following exposure of cells to an HIV antigen. “HIV antigens” include whole (inactivated) virus or any immunogenic components of HIV, e.g., gp120 and p24.

An “intracellular protein” refers to a protein expressed within a cell. Such protein is expressed specifically upon viral infection if the protein is overexpressed in infected cells as compared to a non-infected control cell.

As used herein, the term “anti-viral agent” encompasses natural and synthetic substance.

As used herein, the term “flow cytometry” shall have its art recognized meaning which generally refers to a technique for characterizing biological particles, such as whole cells or cellular constituents, by flow cytometry (See e.g., Jaroszeski et al., Method in Molecular Biology, (1998), vol 91: Flow Cytometry Protocols, Hummama Press; Longobanti Givan, (1992) Flow Cytometry, First Principles, Wiley Liss). All known forms of flow cytometry are intended to be included, particularly fluorescence activated cell sorting (FACS), in which fluorescent labeled molecules are evaluated by flow cytometry.

Methods for performing flow cytometry on samples of immune cells are well known in the art. See e.g., Jaroszeski et al., Method in Molecular Biology, (1998), vol 91: Flow Cytometry Protocols, Hummama Press; Longobanti Givan, (1992) Flow Cytometry, First Principles, Wiley Liss.

A preferred apparatus for performing flow cytometry in the method of the invention is a fluorescence activated cell sorter (FACS). The FACS apparatus commonly includes a light source, usually a laser, and several detectors for the detection of cell particles or subpopulations of cells in a mixture using light scatter or light emission parameters. The underlying mechanisms of FACS are well known in the art, and essentially involve scanning (e.g., counting, sorting by size or fluorescent label) single particles are they flow in a liquid medium past an excitation light source. Light is scattered and fluorescence is emitted as light from the excitation source strikes the moving particle. Forward scatter (FSC, light scattered in the forward direction, i.e., the same direction as the beam) provides basic morphological information about the particles, such as cell size and morphology. Light that is scattered at 90° to the incident beam is due to refracted or reflected light, and is referred to as side angle scatter (SSC). This parameter measures the granularity and cell surface topology of the particles. Collectively, scatter signals in both the forward and wide angle direction are used to identify subpopulations of cells based on cell size, morphology, and granularity. This information is used to distinguish various cellular populations in a heterogeneous sample.

Preparation of Reagents and Cells

Preparation of Dyes

The present invention utilizes a label (radioactive or fluorescent) that is conjugated to an antibody specific to the intracellular protein to effect separation of virally infected viable cells from dead cells. In a preferred embodiment, the label is a fluorophore conjugated to an antibody that specifically binds to a viral protein.

The fluorescent dyes used for detection are greatly determined by the hardware capability of the cytometer. Multi-color laser cytometers exist utilizing some combination of a UV, argon 488, and a HeNe 633 laser (though other combinations exist). Typical color combinations for detection of antibodies are denoted below:

| | |
|---|---|
| 4 color: FITC/PE/PerCP/APC | eg FACSCalibur (BD) |
| 6 color: Above + Cy7PE/Cy7APC | eg FACSVantage (BD) |
| 9 color: Above + cascade blue/DAPI/Cy5PE | eg CYAN (Cytomation) |
| 11 color: Above + Cascade | Custom-Stanford Univ. |
| yellow/Cy5.5PE/TR | |

Most fluorophores are either small organic molecules (FITC, cascade blue, cascade yellow, texas red, DAPI) or fluorescent proteins (PE, PerCP, APC, and Cy-protein tandem conjugates). The small organic dye series from Molecular Probes, the Alexa Fluor dyes, are optimal for intracellular staining, as they are small organic molecules that are easily conjugated and are tolerable to a variety of conditions. Excitation and emission criteria of the hardware will determine which Alexa dye may be used, as substitution of particular dyes is dependent on the end user and the detection system. Species cross reactivity, antibody clone, and source are also things to consider when choosing optimal reagents. The introduction of the tandem conjugate dyes and their subsequent commercialization, has greatly expanded the usage of multicolor applications by the public domain. It should be noted that multiparameter analyses requires appropriate fluorophore compensation that is mediated by both hardware and sometimes software (i.e. 11 colors). Also, the effects, if any, of the experimental conditions on the fluorescent properties of the antibody and other fluorescent reagents need to be assessed prior to their use.

One embodiment of the present invention provides methods for determining the amount of dye incorporated in protein labeling experiments. The ratio of fluorophore to protein ratio may be quantified using dye and protein cross linking kits and a spectrophotometer capable of multiple wavelengths. In general, the degree of labeling may be performed by reading the absorbance value of 280 nm (total protein) and a second absorbance value “x,” which varies depending on the dye, and calculated using the following equations:

(1) Protein concentration calculation:

$$Protein concentration ( M ) = \frac{[ {A}_{280} - ( A 2 \times CF ) ] \times dilution factor}{203 , 000}$$

203,000 is the molar extinction coefficient of a typical IgG (non-IgG proteins will have different molar extinction coefficients

CF is a correction factor to account for dye incorporation at 280 nm

A2 is the absorbance at a specific wavelength for different dyes

(2) Calculate the degree of labeling:

$$Moles dye per mole protein = \frac{A 2 \times dilution factor}{ϵ \times protein concentration ( M )}$$

ε is the molar extinction coefficient of the dye at absorbance value A2.

Spectroscopic properties for dyes are included in the Table 1. In general, 2-10 moles: protein are typical for most dyes, except for dyes that contain PE or APC, where a 1:1 molar ratio is preferred.

TABLE 1
several dyes and relevant spectral properties:

| | | | Molar extinction | Dye |
|---|---|---|---|---|
| | | | coefficient ε at | Correction |
| Dye | λₘₐₓ | Em | λmax | factor at A₂₈₀ |
| Alexa 350 | 346 | 442 | 19,000 | 0.19 |
| Alexa 430 | 434 | 539 | 16,000 | 0.28 |
| Alexa 488 | 494 | 519 | 71,000 | 0.11 |
| Alexa 532 | 530 | 554 | 81,000 | 0.09 |
| Alexa 546 | 558 | 573 | 104,000 | 0.12 |
| Alexa 568 | 577 | 603 | 91,300 | 0.46 |
| Alexa 594 | 590 | 617 | 73,000 | 0.56 |
| Alexa 633 | 632 | 647 | 100,000 | 0.55 |
| Alexa 660 | 663 | 690 | 132,000 | 0.10 |
| Alexa 680 | 679 | 702 | 184,000 | 0.05 |
| Fluorescein | 494 | 518 | 68,000 | 0.20 |
| Cascade Blue | 400 | 420 | 28,00 | 0.65 |
| Rhodamine | 570 | 590 | 120,000 | 0.17 |
| Tetramethylrhodamine | 555 | 580 | 65,000 | 0.30 |
| Texas Red | 595 | 615 | 80,000 | 0.18 |
| Cy5 | 650 | 670 | 250,000 | 0.05 |
| Cy3 | 550 | 570 | 150,000 | 0.08 |
| Cy3.5 | 581 | 596 | 150,000 | 0.24 |
| Cy5.5 | 675 | 694 | 250,000 | 0.18 |
| Cy2 | 489 | 506 | 150,000 | 0.15 |
| R-phycoerythrin (PE) | 566 | 575 | | |
| Allophycocyanin | 655 | 660 | | |
| (APC) | | | | |

Note:
PE and APC conjugates are 1:1
Note:
Tandem conjugates Cy5PE, Cy5.5PE, Cy7PE, Cy5.5, Cy7APC were developed in the Herzenberg laboratory, Stanford University (protocols to be found at www.drmr.com/abcom/index.html) and spectral properties vary from lot to lot because of the resonance energy transfer needed for these special dyes.

Preparation of Antibodies

The present invention utilizes labeled antibodies (e.g. radioactively labeled or fluorescently labeled) specific for an intracellular protein that is expressed specifically upon the viral infection as a reagent to detect virally infected cells. A variety of antibodies specific for such intracellular proteins are available in the art or can be prepared according to conventional antibody production techniques. For detection of virally encoded proteins, antibodies that specifically bind to viral proteins are employed. Such antibodies include but are not limited to antibodies specific for surface or intracellular antigens of Hepatitis A, B, C, and D; antibodies specific for HIV-encoded proteins such as p24, rev, tat, gp120, reverse transcriptase, HIV-1 protease, and HIV-1 integrase; and antibodies directed to Herpes and other viral proteins.

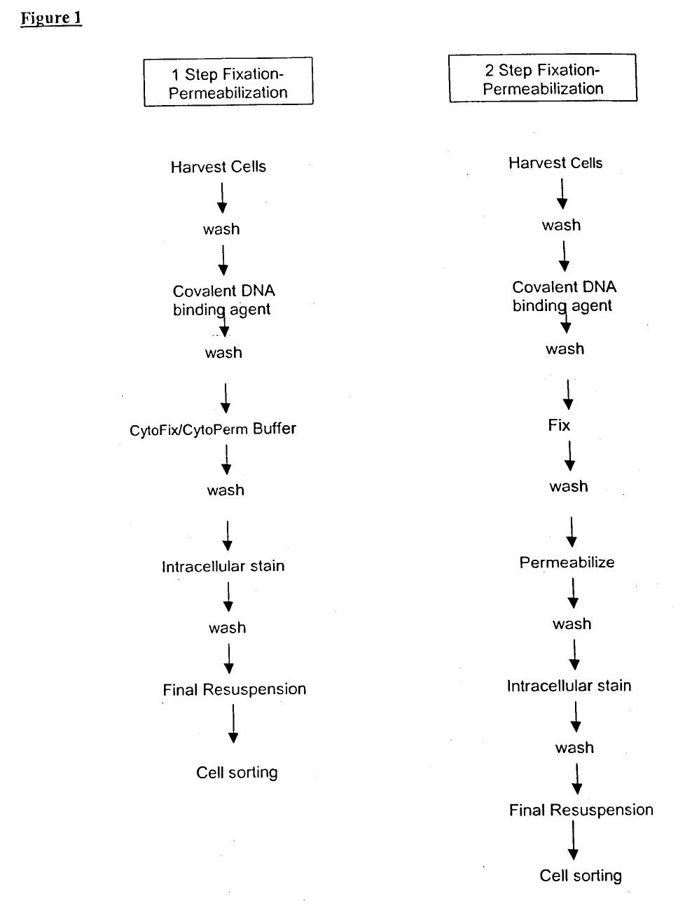

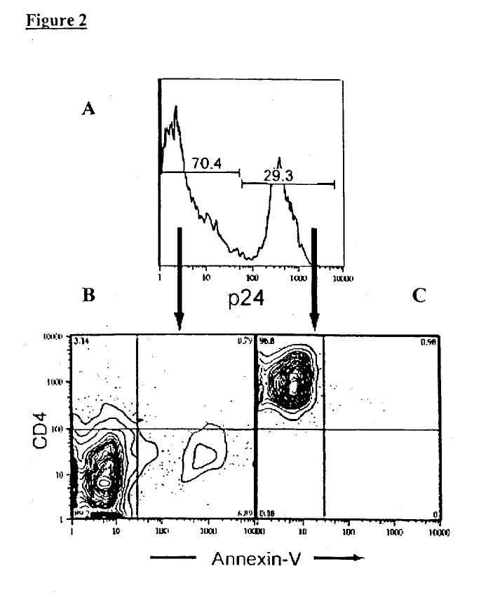

The antibodies embodied by the present invention can either be monoclonal or polyclonal. Both directly conjugated antibodies, and two-step staining procedures (i.e. primary+fluorophore labeled secondary) (see FIG. 1) can be used for surface stain. Specificity is routinely tested by in vitro based immunoblot analysis using both purified and recombinant proteins. Surface staining procedures may implement a blocking agent, either fetal calf serum or bovine serum albumin, to eliminate non-specific binding. If considering non-specific Fc receptor staining, Fc receptor blocking reagents are commercially available. Fab fragments have also been used to this extent. Cross reactivity amongst species by antibodies may be tested by either the manufacturer or producer of the reagent.

Antibodies recognizing the viral protein (e.g. HIV p24 protein) may be conjugated to the different fluorescent dyes. Commercially available antibodies are available on several colors. Particular colors, however, such as the Cy-tandem conjugates, the Alexa Fluor dyes, and some protein-fluorphore dyes need to be self-conjugated. Antibodies obtained through commercial vendors may be spin dialyzed of high azide contents (1 mM azide is permitted for conjugations) and stabilizing agents such as BSA. A recommended source is Amicon's centricon protocols for performing antibody buffer exchanges (PBS, pH 7.4). Also, the concentration for optimal conjugation may be achieved by this method. In a preferred embodiment, 500-1000 μg is suited for conjugations and subsequent testing.

Antibody titrations may be necessary to determine the optimal concentration of antibody necessary for staining a fixed number of cells. The objective being to obtain the highest signal to noise without compromising detection or specificity. Fluorophore compensation may be critical when working with multiple colors to eliminate fluorophore emission bleed-through in channels designated for different fluorophores. In a preferred embodiment, antibodies used for either surface or intracellular staining can be used in cocktails once an optimal concentration has been determined. A cocktail of antibodies specific for intracellular proteins can be used so long as they do not interfere with each other.

A variety of commercial kids are available for detecting antibody stains. A preferred kit is tyramide signal amplification kit supplied by Molecular Probes.

Preparation of Cells

To prepare the cell sample, cells are first stimulated and harvested as needed. In a preferred embodiment, mononuclear cells may be isolated from blood of healthy donors. Human peripheral blood monocytes may be obtained by Ficoll-plaque density centrifugation (Amersham Pharmacia, Uppsala, Sweden) of whole blood and depletion of adherent cells by adherence to plastic culture dishes. Cells may then be activated with human recombinant IL-2 for 24 hours prior to HIV-1 infection. Treatment samples may be synchronized for time, and processed simultaneously. All culture reagents may be replenished every 3 days. Quantitative cell counts may be obtained using TruCount beads (Becton Dickinson Biosciences).

Once stimulated, primary cells may be harvested in 15 ml conical tubes, washed one time with an ice cold washing buffer (2-5 mls adequate). The cells may then be spun at 1500 rpm and 4 degrees. Adherent cells may be harvested with the washing buffer outlined, by washing cells grown in 12 well plates with 500 μL washing buffer, incubating in 500 μL washing buffer for 5 minutes, and pipetting gently to loosen cells into a single cell suspension. The isolated cells may be maintained in complete media (RPMI-1640, 10% FCS, 1% PSQ) at 37° C. and 5% CO₂.

In one embodiment, the HIV-1 strains were referred to as R5, X4, or R5X4 depending on the co receptor used for viral entry. Virus containing supernatants were harvested 3, 6, 9, 12 days and stored at −80° C. TCID₅₀ was determined in IL-2 stimulated PBMC. After culturing for 24 hours in IL-2, the cells were then infected by a 2 hour incubation with HIV-1_{BaL} at two doses (1500 TCID₅₀/1×10⁶ cells, and 300 TCID₅₀/1×10⁶ cells). Every 3 days, cells may be split and replenished with all stimuli. Cell free supernatants may be saved for p24 and RT activity assays and cells were processed for flow cytometry. Supernatants from HIV-1 infected and treated cultured cells may be subjected to a p24 ELISA as described by the manufacturer of the p24 ELISA kit (NEN). The cells were then washed with a washing buffer, such as phosphate buffered saline, pH 7.4 with 0.5 mM EDTA and 2.5 mM Na₂PO₄.

Staining:

A central feature of the present invention is the use of a covalent nucleic acid binding agent such as membrane exclusion dye that covalently binds to nucleic acids such as DNA. Preferred covalent nucleic acid binding agents include but are not limited to ethidium monoazide (EMA) and actinomycin D. The covalent nucleic acid binding agents stain preferably dead cells because membranes of the dead cells are more susceptible to the penetration of these agents. Specifically, EMA is an ethidium bromide analogue that is excluded by intact cellular membranes, and forms a covalent adduct with DNA upon a pulse of light. After EMA staining, cells are fixed and permeabilized to permit an antibody capable of detectably labeling the target antigen to traverse the plasma membrane into the cytoplasm of the cell. Subsequent permeabilization does not affect this compound, making it a superior discriminator of live and dead cells. Differentiating the dead cells from viable cells is of great significance when working with cell populations that comprise less than 10% of the total cell population (i.e., lymphocyte subsets within PBMC).

After contact with a covalent nucleic acid binding agent such as EMA, the invention employs fixation and permeabilization steps to allow intracellular staining. Fixation may occur using a low percentage paraformaldehyde treatment (<2%) (PFA). A minimum of a final 0.5% PFA may be required, but a 1-2% PFA is optimal. Greater than 4% PFA will induce cellular aggregates and obstruct the fluidic system of the cytometer. Permeabilization conditions were found to be optimal by using a saponin based buffer. The use of harsh detergents may be detrimental to the antibody reagents. Also, too high of detergent concentration was detrimental to the fluorescent properties of the protein-fluorophores. Individual testing of fixation and permeabilization conditions may be determined for the reagents being used prior to intracellular staining. Fixation procedures using alcohol fixatives are likely not suited for this application. Intracellular p24 staining may be achieved by first directly conjugating a human anti-p24 mAb antibody to Alexa Fluor 488 (Molecular Probes, Or). The cells are then suspended in an intracellular staining cocktail. A cocktail of antibodies specific for intracellular proteins of interest may be used so long as they do not interfere with each other. The stained cells are then washed, resuspended in the fixative buffer, and transferred to a FACS tube for analysis. As another expansion of the inventive embodiment, intracellular staining can be combined with surface staining.

In another embodiment of the present invention, cells may be suspended in ice-cold buffer (50 mL for 1-2×10⁶ cell). The cells are then incubated with the surface cocktail containing EMA and an extracellular staining buffer, such as deficient RPMI, 4% FCS and 0.001% azide for approximately 15 minutes on ice. This procedure should minimize exposure to light. After approximately 15 minutes, the cells are subjected to multiple washes in the washing buffer (phosphate buffered saline, pH 7.4 with 0.5 mM EDTA and 2.5 mM Na₂PO₄) and resuspended. The incubated cells are then pulsed with light for 1-5 minutes. After staining with covalent nucleic acid binding agent, the cells may be fixed with a fixative buffer, such as 1% paraformaldehyde in PBS, on ice for approximately 30 minutes in the dark. Wash buffer may be added, and the cells may be pelleted. Permeabilization may occur by pipetting up and down with a permeabilization buffer, such as 0.1% saponin with 4% FCS in deficient RPMI. The cells may be incubated for about 30 minutes at 4 deg in the dark and subsequently washed again. Intracellular staining is performed by suspending the cells for 30 minutes on ice in the dark in an intracellular staining cocktail made up in a permeabilization buffer, such as 0.1% saponin with 4% FCS in deficient RPMI. The cells are then washed and transferred to a FACS tube for analysis.

Where desired, staining control can be employed in all flow cytometry applications. Both positive and negative cell populations for the parameter of interest are needed to properly analyze samples, and adjust compensation parameters. Single color controls and isotype controls are recommended when performing multi-color experiments. Unlabeled controls may be used for autofluorescence. Intracellular isotope controls may be used for background staining. Hardware settings, such as PMT voltages and compensation percentages should be verified to be accurate if using saved settings, as they often need to be readjusted).

The methods of the present invention have optimized the protocols for suspension cells, although have had success with adherent fibroblast such as NIH3T3 for intracellular phospho-staining. Adherent cells need to be removed from the plate using a PBS/EDTA solution, and not trypsinized or scraped off because to do so will lose antigen detection. For NIH3T3 cells, cell permeable phosphatase inhibitors in the PBS/EDTA buffer greatly enhanced phospho-detection. Washing and centrifugation steps can affect signaling systems within cells and should be determined upon an experimental basis if considered a concern. However, the detection is made on a relative (stimulated to unstimulated cells) and not absolute scale.

The power of these techniques may be most appreciated in multiparameter analyses where both surface and intracellular staining conditions are combined. Combining intracellular proteins and surface stains requires stepwise considerations for all the reagents and experimental details necessary. Other parameters such as cell cycle, apoptosis, and physiological readouts (calcium levels, redox, pH, membrane potential) can also be combined, however, each parameter requires additional considerations when combined with the staining methods described here. Transport rates and proper fixation are considerations for use of small fluorescent chemicals as sensors for detection of intracellular events.

The following example is meant to illustrate, but not to limit, the methods of the invention. Modifications of the conditions and parameters set forth below that are apparent to one skilled in the art are included in the invention.

### EXAMPLES

The methods of the present invention were used to analyze the response of HIV-infected CD4⁺ cells in IL-2 stimulated cultures in vitro to motexafin gadolinium (Gd-Tex). Gd-Tex is a compound that promotes intracellular oxidative stress and has been reported to localize tumors and to enhance radiation response in animal tumor models.

Peripheral blood mononuclear cells (PBMC) isolated from healthy donors were first activated in culture with recombinant human IL-2 and infected in vitro by HIV. The isolated cells were maintained in complete media (RPMI medium 1640, 10% (vol/vol) FCS, 1% (vol/vol) PSQ) at 37° C. and 5% CO₂. Cells were activated with human recombinant IL-2 for 24 hours prior to HIV-1 infection. BSO treatments were preformed at 5 mM for 72 hours and N-acetylcysteine (NAC) treatments were performed at 5 mM for 24 hours. NAC alleviated Gd-Tex toxicity at high Gd-Tex concentrations. The BSO treatment rendered PBMC more sensitive to killing with Gd-Tex. Treatment samples were synchronized for time, and processed simultaneously. All culture reagents were replenished every 3 days. Quantitative cell counts were obtained using TruCount beads (BD Biosciences).

After isolation and activation, the cells were infected in vitro with HIV-1 at MOI of 30 to 150. The HIV-1 strains were referred to as R5, X4, or R5X4 depending on the co receptor used for viral entry. The M-tropic R5 prototype strain (BaL) was used in these studies and primary isolates were obtained from the National Institutes of Health AIDS reagent program. Virus containing supernatants were harvested 3, 6, 9, 12 days and stored at −80° C. TCID₅₀ was determined in IL-2 stimulated PBMC. Cells were cultured for 24 hours in IL-2, then infected by a two hour incubation with HIV-1_{BaL} at two doses (1500 TCID₅₀/1×10⁶ cells, and 300 TCID₅₀/1×10⁶ cells). Every 3 days, cells were split and replenished with all stimuli. Cell free supernatants were saved for p24 and RT activity assays and cells were processed for flow cytometry. The supernatants from HIV-1 infected and treated cultured cells were subjected to p24 ELISA. p24 levels were monitored at 3 day intervals and quantified using a p24 standard curve prepared with recombinant p24. Reverse transcriptase assays were performed by Reverse transcriptase activity assay kit (Molecular Probes) according to the manufacturer's instructions. Approximately 1-10×10⁷ peripheral blood mononuclear cells were treated with IL-2 (100 U/ml) for 24 hours and subsequently treated with Gd-Tex, NAC, or BSO and prepared for flow cytometry. Extracellular and intracellular staining were performed as described in the previous section. Cells were surface stained in an extracellular staining buffer containing deficient RPMI, 4% FCS, and 0.001% azide and then stained for 20 minutes using pre-titred antibodies (0.1-0.8 ug of ab/1×10⁶ cells). Cells were fixed and resuspended in a fixing buffer (1% paraformaldehyde in PBS). Intracellular p24 staining was achieved by directly conjugating a human anti-p24 mAb antibody to Alexa Fluor 488 (Molecular Probes). In between washes were performed in phosphate buffered saline wash (pH 7.4 and 0.5 mM EDTA). Isotype control match antibodies were used for all antibodies. Eleven-color data acquisition was collected on a modified FACStarPlus (Becton Dickinson, San Jose, Calif.) connected to MoFlo electronics (Cytomation, Fort Collins, Colo.). The Gd-Tex treated cells were then analyzed by FACS analysis of intracellular HIV or p24 and concomitant surface marker expression. The method enabled quantitative measurements of apoptosis in HIV-1 infected CD4+ T cells.

Analysis by the methods of the present invention suggested that in vitro HIV infection depletes glutathione (GSH) in PBMC cultures. FACS analysis showed that more than 98 percent of CD4+ cells harvested 6 days after infection were producing virus and that virus production did not in and of itself induce apoptosis under these conditions. Concomitant analysis of GSH with the monochlorobimane assay demonstrated that even at the lowest HIV dose tested, GSH levels in the infected cells were decreased roughly eight-fold for CD4 T cells and 2 fold for co-resident CD8 T cells. This HIV-infection mediated GSH depletion did not appear to be highly detrimental since it did not result in apoptosis induction and did not decrease the cell yield relative to uninfected cultures. Since GSH levels did not drop more than 10 percent in uninfected cultures (data not shown), the GSH decrease in CD8 T cells in the infected cultures must have been a consequence of the infection. Most likely, it represented the GSH depleting activity of HIV-TAT, which is known to be released in HIV-infected cultures.

At high-doses, Gd-Tex depleted GSH and was toxic to T cells in PBMC. At Gd-Tex doses above 400 uM, nearly all IL-2 activated PBMC T cells died within 24 hours when cultured in the presence of 1 mM Gd-Tex. However, at Gd-Tex doses below 400 uM, toxicity was substantially decreased. At doses below 250 uM, toxicity was essentially undetectable. Consistent with previous indication, Gd-Tex toxicity in PBMC was due to GSH depletion and the consequent induction of oxidative stress. At lower-doses, Gd-Tex selectively killed HIV-infected CD4 T cells. The same doses did not kill uninfected CD4 T cells and CD8 T cells. At Gd-Tex doses below 250 □M, GSH depletion and Gd-Tex toxicity in CD8 T cells and uninfected CD4 T cells was minimal. However, for HIV-infected cells, even 3 uM Gd-Tex selectively killed HIV-infected (p24+) CD4 T cells. These findings are shown in FIG. 4-5, in which data are shown for subset-defining cell surface marker expression, HIV infection, intracellular GSH and Gd-Tex toxicity (induction of apoptosis and the breaking of the cell permeability barrier), all measured simultaneously for individual cells by 11-color, 13-parameter Hi-D FACS.

The mechanism responsible for this selective killing does not solely appear to depend on induction of oxidative stress. Although HIV may deplete GSH, this depletion is not as marked by GSH depletion caused by high dose Gd-Tex. Furthermore, it occurs equally in CD4 and CD8 T cells, whereas the low-dose Gd-Tex selectively kills CD4 T cells. In fact, low dose Gd-Tex only kills HIV-infected CD4 T cells that are propagating the virus, as determined by the p24 stain, suggesting that HIV replication is itself in some way required to enable low-dose Gd-Tex toxicity.

Based on the methods of the present invention, the results indicated that Gd-Tex selectively induced apoptosis in HIV-1 infected CD4 T cells. Importantly, this occurred at Gd-Tex concentrations that are not cytotoxic to uninfected cells in the culture. These findings suggest that Gd-Tex may have therapeutic utility as a novel anti-HIV agent capable of selectively targeting and removing HIV-1 infected cells in an infected host.

What is claimed is:

1. A method of separating virally infected viable cells from dead cells, comprising:
(i) providing a population of cells from an individual suspected of having a viral infection;
(ii) staining the population of cells with a covalent nucleic acid binding agent;
(iii) contacting the population of cells of step (ii) with a labeled antibody specific for an intracellular protein that is expressed specifically upon viral infection of the cells, under conditions suitable for a specific binding of the antibody to the intracellular protein within a cell; and
(iv) separating the population of cells of step (iii) to obtain a plurality of cells that are not stained with the covalent nucleic acid agent but are labeled with the antibody, and/or to obtain a separate population of cells that are stained with the nucleic acid binding agent.

2. The method of claim 1, wherein the individual is suspected of having an HIV infection.

3. The method of claim 1, wherein the individual is suspected of having a Herpes virus or Ebola infection.

4. The method of claim 1, wherein the individual is suspected of having a Hepatitis virus infection.

5. The method of claim 4, wherein the virus is Hepatitis A.

6. The method of claim 4, wherein the virus is Hepatitis B.

7. The method of claim 4, wherein the virus is Hepatitis C.

8. The method of claim 4, wherein the virus is Hepatitis D.

9. The method of claim 1, wherein the nucleic acid binding agent is ethidium monoazide (EMA).

10. The method of claim 1, wherein the nucleic acid binding agent is actinomycin D.

11. The method of claim 1, wherein the antibody is fluorescently labeled.

12. The method of claim 1, wherein the antibody is radioactively labeled.

13. The method of claim 1, wherein the intracellular protein is encoded by the virus.

14. The method of claim 1, wherein the intracellular protein is a host cell protein overexpressed in response to the viral infection.

15. The method of claim 1, wherein the plurality of cells that are stained with the covalent DNA are dead cells.

16. The method of claim 1, wherein the plurality of cells that are not stained with the nucleic acid binding agent but labeled with the antibody are virally infected viable cells.

17. The method of claim 1, wherein the separation is effected by a cell sorting process.

18. The method of claim 1, wherein the cell sorting process is fluorescence activated cell sorting (FACS).

19. The method of claim 1, wherein the cells are animal cells.

20. The method of claim 19, wherein the cells are mammalian cells.

21. A method of assessing HIV infection and/or replication in viable cells in an individual suspected of having an HIV viral infection, comprising:
(i) providing a population of cells from the individual;
(ii) staining the population of cells with a covalent nucleic acid binding agent;
(iii) contacting the population of cells of step (ii) with a labeled antibody specific for an intracellular protein that is expressed specifically upon viral infection of the cells, under conditions suitable for a specific binding of the antibody to the intracellular protein within a cell; and
(iv) separating the population of cells of step (iii) to obtain a plurality of cells that are not stained with the covalent nucleic acid binding agent but are labeled with the antibody, thereby assessing HIV infection of viable cells in the individual.

22. The method of claim 21, further comprising the step of obtaining a separate population of cells that are stained with the covalent nucleic acid binding agent.

23. The method of claim 21, wherein the covalent nucleic acid binding agent is ethidium monoazide (EMA).

24. The method of claim 21, wherein the covalent nucleic acid binding agent is actinomycin D.

25. The method of claim 21, wherein the antibody is fluorescently labeled.

26. The method of claim 21, wherein the antibody is radioactively labeled.

27. The method of claim 21, wherein the intracellular protein is encoded by the virus.

28. The method of claim 21, wherein the intracellular protein is a host cell protein overexpressed in response to the viral infection.

29. The method of claim 21, wherein the plurality of cells that are stained with the covalent DNA are dead cells.

30. The method of claim 21, wherein the separation is effected by a cell sorting process.

31. The method of claim 21, wherein the cell sorting process is fluorescence activated cell sorting (FACS).

32. The method of claim 21, wherein the cells are animal cells.

33. The method of claim 32, wherein the cells are mammalian cells.

34. The method of claim 21, wherein the intracellular protein is an HIV protein selected from the group consisting of p24, rev, tat, gp 120, reverse transcriptase, HIV-1 protease, and HIV-1 integrase.

35. The method of claim 21, wherein the cells are peripheral blood mononuclear cells.

36. A method of monitoring the effectiveness of an anti-viral therapy comprising assessing viral infection and/or replication in viable cells in an individual, wherein a reduction in viral infection and/or replication is indicative of the effectiveness of the therapy, wherein the assessment comprises the steps of:
(i) providing a population of cells from an individual infected with the virus;
(ii) staining the population of cells with a covalent nucleic acid binding agent;
(iii) contacting the population of cells of step (ii) with a labeled antibody specific for an intracellular protein that is expressed specifically upon viral infection of the cells, under conditions suitable for a specific binding of the antibody to the intracellular protein within a cell; and
(iv) separating the population of cells of step (iii) to obtain a plurality of cells that are not stained with the covalent nucleic acid binding agent but are labeled with the antibody, thereby assessing viral infection of viable cells in the individual.

37. The method of claim 36, wherein the cells are peripheral blood mononuclear cells.

38. The method of claim 36, wherein the viral infection is mediated by a virus selected from the group consisting of HIV, Herpes, Hepatitis A, Hepatitis B, Hepatic C and Hepatitis D.

39. A method of identifying an anti-viral agent, comprising assessing viral infection and/or replication in viable cells in an individual infected with the virus, wherein a reduction in viral infection and/or replication upon contacting a candidate anti-viral agent with a population of cells from the individual is indicative of the identification of an anti-viral agent, wherein the assessment of the viral infection and/or replication in viable cells in the individual comprises the steps of:
(i) providing a population of cells from the individual;
(ii) staining a population of cells obtained from the individual of step (i) with a covalent nucleic acid binding agent;
(iii) contacting the population of cells of step (ii) with a labeled antibody specific for an intracellular protein that is expressed specifically upon viral infection of the cells, under conditions suitable for a specific binding of the antibody to the intracellular protein within a cell; and
(v) separating the population of cells of step (iii) to obtain a plurality of cells that are not stained with the covalent nucleic acid binding agent but are labeled with the antibody, thereby assessing viral infection of viable cells in the individual.

40. The method of claim 39, wherein the cells are peripheral blood mononuclear cells.

41. The method of claim 39, wherein the viral infection is mediated by a virus selected from the group consisting of HIV, Herpes, Hepatitis A, Hepatitis B, Hepatic C and Hepatitis D.
